(19) United States
(12) Patent Application Publication
(10) Pub. No.: US 2010/0212058 A1
(43) Pub. Date: Aug. 26, 2010

(54) TILTABLE WELDING HELMET

(75) Inventor: Daniel J. Wanhainen, Macedonia, OH (US)

Correspondence Address: Lincoln Electric Company/Perkins COIE LLP, 607 Fourteenth Street , NW, Washington, DC, 20005-2003, US

(73) Assignee: Lincoln Global, Inc., Industry, CA (US)

(21) Appl. No.: 12/392,503

(22) Filed: Feb. 25, 2009

(51) Int. Cl. A61F 9/06; A42B 1/08

(52) U.S. Cl. 2/8.2; 2/424

(57) ABSTRACT

A protective helmet comprising having a protective shell, and a headgear mounted to the protective shell, wherein the headgear includes a tilt mechanism, the tilt mechanism comprising a tilt arm having a spring secured to the tilt arm, the spring having free portion disposed between a first portion and a second portion, the first portion and second portion being secured to the tilt arm and the free portion being free from the tilt arm.

### BACKGROUND OF THE INVENTION

1. Field of the Invention

Devices and apparatuses consistent with the present invention relate to tilt control mechanisms for protective helmets, including welding helmets.

2. Description of the Related Art

Welders often wear welding helmets with a protective darkened window to prevent injury to the welder's face and eyes during the welding operation. Such welding helmets often have tilt mechanisms that allow the welder to tilt the protective shell portion of the welding helmet between an up position, i.e., to place the protective shell portion so that it is no longer in front of the welder's face, allowing the welder a relatively unobstructed view of a workpiece, and a down position, i.e., the protective position.

Conventional welding helmets have a problem in that conventional tilt mechanisms do not allow the welder to adjust the position of the protective shell portion of the welding helmet when tilted, i.e., the protective shell's degree of rotation, and do not sufficiently secure the shell portion when in the up position, allowing the protective shell portion to return to a down position unexpectedly. Such unexpected movement is aggravated over time as the welder tilts the welding helmet between an up position and down position with high frequency, causing wear on the components used in conventional tilt mechanisms. Thus, there is a need in the art for a welding helmet having an improved tilting mechanism.

### BRIEF SUMMARY OF THE INVENTION

The presently disclosed invention includes a protective helmet comprising a protective shell, and a headgear mounted to the protective shell, wherein the headgear includes a tilt mechanism, the tilt mechanism comprising a tilt arm having a spring secured to the tilt arm, the spring having a free portion disposed between a first portion and a second portion, the first portion and second portion being secured to the tilt arm and the free portion being free from the tilt arm.

The invention also comprises a tilt mechanism for use in protective helmets, the protective helmet having a protective shell and a headgear mounted to the protective shell with the tilt mechanism mounted to the headgear, the tilt mechanism comprising a tilt arm; and a spring secured to the tilt arm, wherein the spring has free portion disposed between a first portion and a second portion, the first portion and second portion being secured to the tilt arm and the free portion being free from the tilt arm.

### BRIEF DESCRIPTION OF THE DRAWINGS

The above and/or other aspects of the invention will be more apparent by describing in detail exemplary embodiments of the invention with reference to the accompanying drawings, in which:

FIG. 1 illustrates a welding helmet with the protective shell portion in both a down position and in an up position.

FIG. 2 illustrates a rear view of a welding helmet and illustrates the headgear of the welding helmet.

FIG. 3 illustrates a first view of a tilt arm in accordance with an embodiment of the present invention.

FIG. 4 illustrates a perspective view of the tilt arm of FIG. 3.

FIG. 5 illustrates a view of the tilt angle adjustment member.

### DETAILED DESCRIPTION OF EXEMPLARY EMBODIMENTS

Exemplary embodiments of the invention will now be described below by reference to the attached figures. The described exemplary embodiments are intended to assist the understanding of the invention, and are not intended to limit the scope of the invention in any way. Like reference numerals refer to like elements throughout.

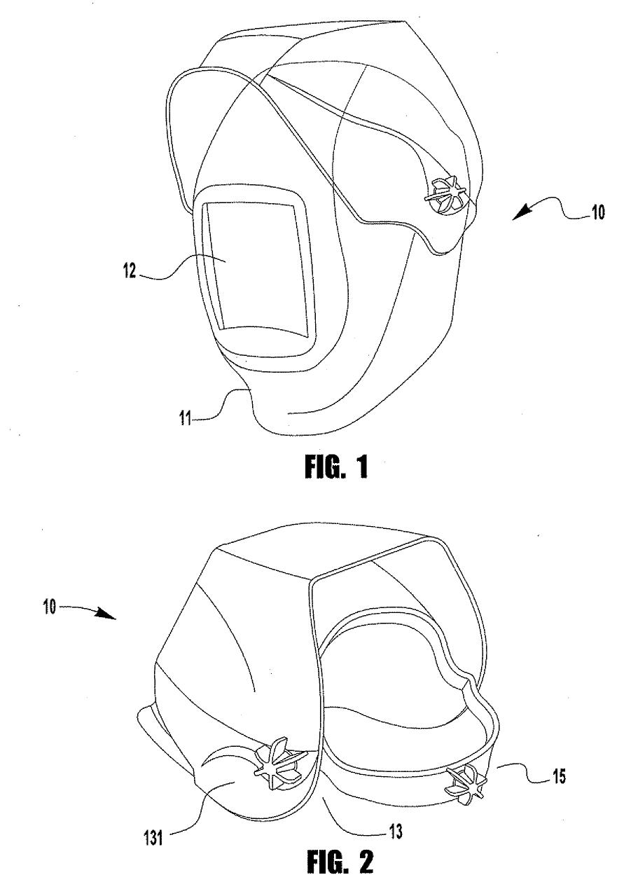

FIG. 1 depicts a diagrammatical representation of an exemplary embodiment of welding helmet 10 in the down position and in the up position. The down position is the position a welder would place the helmet 10 while welding. The up position is a position a welder would place the helmet 10 when the welder is not welding.

Although this invention is described with respect to welding helmets, the scope of the invention is not limited to welding helmets. The invention described herein can be applied to any type of protective helmet.

As can be seen in FIG. 1, welding helmet 10 includes a protective shell portion 11. Protective shell portion 11 is designed to protect the welder's face from sparks and debris, and to protect the welder's eyes from light from the welding operation. Integral to protective shell portion 11 is a protective lens 12. A variety of protective lenses are known in the art. Protective lens 12 can be any type of lens, including an auto-darkening lens.

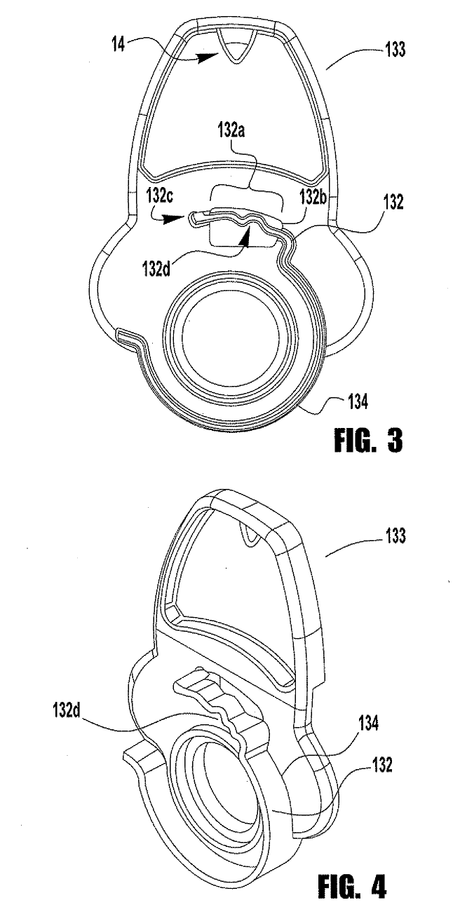

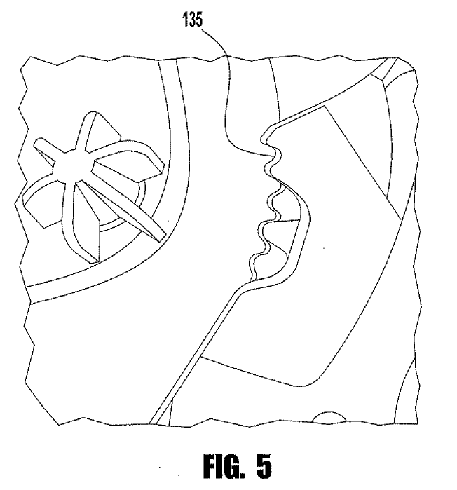

FIG. 2 shows a rear view of a welding helmet 10. As illustrated in FIG. 2, a welding helmet 10 includes a headgear 15 and a tilt mechanism 13. A welder is able to place welding helmet 10 in an up position without removing welding helmet 10 due to a tilt mechanism 13. Headgear 15 comprises any number of straps, brackets and other structures to allow the helmet 10 to securely fit onto a welder's head. A wide array of headgear types and styles are known in the art, and the present invention is not limited in this regard.

As shown in FIGS. 2 and 3, an exemplary embodiment of the tilt mechanism 13 comprises a knob 131 for securing the headgear to the shell portion 11 and for controlling the rate of movement of the shell portion 11 between the up and down positions, a spring 132, and a tilt arm 133. Spring 132 is securely attached to tilt arm 133. In one exemplary embodiment, spring 132 is located in a trough 134 located in tilt arm 133.

Spring 132 has a free portion 132a. Free portion 132a is not secured to tilt arm 133 and is able to deflect. Both end 132b and 132c of spring 132 are secured to tilt arm 133. The securing of end 132c to tilt arm 133 has not previously been performed in the prior art.

The spring 132 and ends 132b and 132c can be secured to the arm 133 by any known means. For example, the spring 132 can be molded into the arm 133. Alternatively, the spring 132 can be secured via an adhesive or mechanical means, or can be integrally formed with the arm 133.

In exemplary embodiments of the present invention, spring 132 can be made from metal or hard plastic materials.

Further, it is noted that the present invention is not intended to be limited to the configuration and appearance shown in the figures. The figures are merely intended to be exemplary. For example, in the embodiment shown, the spring 132 curves around the bottom of the arm 133 as shown, and arm 133 has an open portion positioned adjacent to the free portion 132a. However, the present invention is not so limited.

In an exemplary embodiment, spring 132 includes a detent 132d. Detent 132d is designed to cooperatively engage a protrusion on headgear 15. The protrusion is located on headgear 15 such that the protrusion engages detent 132d when welding helmet 10 is rotated to the up position. When the helmet 10 moves from a down position to an up position, the protrusion first encounters spring 13 near end 132c. Because spring 13 is fixed at both ends 132b and end 132c, the protrusion is firmly held in detent. The fixing of both end 132b and end 132c allows the welding helmet 10 to remain in the up position until a welder desires the welding helmet 10 to be placed in a down position. A welder can easily move the helmet 10 from an up position to a down position. During this operation, the force of the welder urging welding helmet 10 into a down position moves protrusion out of detent, allowing the helmet 10 to rotate into the down position. Because end 132c is fixed to tilt member 13, the tilt mechanism 13 maintains its ability to hold helmet 10 in an up position even after prolonged use. Further, additional force is required to move the helmet 10 over prior known systems thus aiding in the prevention of inadvertent moving of the helmet 10.

Tilt arm 133 can also include a tilt angle adjustment member 14. Tilt angle adjustment member 14 is configured to engage one or more tilt angle adjustment slots 135 located on the protective portion 11. A welder can move the tilt angle adjustment member 14 into any of the tilt angle adjustment slots 135, thereby changing the viewing angle and the helmet 10 down stop, i.e., the position of the helmet 10 in the down position.

Implementing various embodiments of the welding helmet 10 and its tilt mechanism 13 are well within the capabilities of those of ordinary skill in the art.

While the invention has been particularly shown and described with reference to exemplary embodiments thereof, the invention is not limited to these embodiments. It will be understood by those of ordinary skill in the art that various changes in form and details may be made therein without departing from the spirit and scope of the invention as defined by the following claims.

What is claimed is:

1. A protective helmet comprising:
a protective shell, and
a headgear mounted to the protective shell, wherein the headgear includes a tilt mechanism, the tilt mechanism comprising a tilt arm having a spring secured to the tilt arm, the spring having free portion disposed between a first portion and a second portion, the first portion and second portion being secured to the tilt arm and the free portion being free from the tilt arm.

2. The protective helmet according to claim 1 wherein the free portion includes a detent configured to engage a protrusion to allow the protective helmet to be rotated from a down position to an up position and to be held in the up position.

3. The protective helmet according to claim 2 wherein the tilt arm includes a trough for receiving the spring.

4. The protective helmet according to claim 3 wherein the tilt arm includes a tilt angle adjustment member, the tilt angle adjustment member comprising a protrusion for adjustably engaging one of a plurality tilt angle adjustments slots disposed on the helmet.

5. A tilt mechanism for use in protective helmets, the protective helmet having a protective shell and a headgear mounted to the protective shell with the tilt mechanism mounted to the headgear, the tilt mechanism comprising:
a tilt arm; and
a spring secured to the tilt arm, wherein the spring has free portion disposed between a first portion and a second portion, the first portion and second portion being secured to the tilt arm and the free portion being free from the tilt arm.

6. The tilt mechanism according to claim 5 wherein the free portion includes a detent configured to engage a protrusion to allow the protective helmet to be rotated from a down position to an up position and to be held in the up position.

7. The tilt mechanism according to claim 6 wherein the tilt arm includes a trough for receiving the spring.

8. The tilt mechanism according to claim 7 wherein the tilt arm includes a tilt angle adjustment member, the tilt angle adjustment member comprising a protrusion for adjustably engaging one of a plurality tilt angle adjustments slots disposed in the helmet.
